(19) United States
(12) Patent Application Publication
(10) Pub. No.: US 2019/0070001 A1
(43) Pub. Date: Mar. 7, 2019

(54) MEDICAL DEVICE WITH TIP MEMBER

(71) Applicant: BOSTON SCIENTIFIC SCIMED, INC., MAPLE GROVE, MN (US)

(72) Inventors: Michael P. Calomeni, San Jose, CA (US); Mimi Trinh Fitterer, Belmont, CA (US)

(73) Assignee: BOSTON SCIENTIFIC SCIMED, INC., MAPLE GROVE, MN (US)

(21) Appl. No.: 16/115,941

(22) Filed: Aug. 29, 2018

Related U.S. Application Data
(60) Provisional application No. 62/554,258, filed on Sep. 5, 2017.

(51) Int. Cl. A61F 2/24

(52) U.S. Cl. CPC A61F 2/2436; A61F 2002/9665

(57) ABSTRACT

Medical devices and methods for making and using medical devices are disclosed. An example system medical device delivery system includes an outer shaft having a proximal end region, a distal end region and a lumen extending therein. The delivery system also includes an inner shaft having a proximal end region, a distal end region and a lumen extending therein, wherein the inner shaft extends within at least a portion of the lumen of the outer shaft. The delivery system also includes a tip member disposed the along the distal end region of the outer shaft, the tip member including a proximal end region and a distal end region; wherein the outer shaft and the inner shaft are configured to position an implantable medical device therebetween and wherein the tip member includes an annular groove positioned adjacent the proximal end region of the tip member.

### CROSS-REFERENCE TO RELATED APPLICATIONS

This application claims the benefit of priority under 35 U.S.C. § 119 to U.S. Provisional Application Ser. No. 62/554,258, filed Sep. 5, 2017, the entirety of which is incorporated herein by reference.

### TECHNICAL FIELD

The present disclosure pertains to medical devices, and methods for manufacturing medical devices. More particularly, the present disclosure pertains to medical devices including a tip member designed to limit deformation of the outer shaft of the medical device.

### BACKGROUND

A wide variety of intracorporeal medical devices have been developed for medical use, for example, intravascular use. Some of these devices include guidewires, catheters, and the like. These devices are manufactured by any one of a variety of different manufacturing methods and may be used according to any one of a variety of methods. Of the known medical devices and methods, each has certain advantages and disadvantages. There is an ongoing need to provide alternative medical devices as well as alternative methods for manufacturing and using medical devices.

### BRIEF SUMMARY

This disclosure provides design, material, manufacturing method, and use alternatives for medical devices. An example system medical device delivery system includes an outer shaft having a proximal end region, a distal end region and a lumen extending therein. The delivery system also includes an inner shaft having a proximal end region, a distal end region and a lumen extending therein, wherein the inner shaft extends within at least a portion of the lumen of the outer shaft. The delivery system also includes a tip member disposed the along the distal end region of the outer shaft, the tip member including a proximal end region and a distal end region; wherein the lumen of the outer shaft is configured to position an implantable medical device therein and wherein the tip member includes an annular groove positioned adjacent the proximal end region of the tip member.

Alternatively or additionally to any of the embodiments above, wherein at least a portion of the distal end region of the outer shaft is designed to engage the annular groove.

Alternatively or additionally to any of the embodiments above, wherein the annular groove is proximally facing.

Alternatively or additionally to any of the embodiments above, wherein the annular groove has a first profile and wherein the distal end region of the outer shaft has a second profile, and wherein the first profile is configured to match the second profile.

Alternatively or additionally to any of the embodiments above, wherein a portion of the tip covers at least a portion of the distal end region of the outer shaft.

Alternatively or additionally to any of the embodiments above, wherein a portion of the proximal end region of the tip member is configured to extend within at least a portion of the lumen of the outer shaft.

Alternatively or additionally to any of the embodiments above, wherein the implantable medical device includes an implantable heart valve.

Alternatively or additionally to any of the embodiments above, wherein the tip member further includes a lumen extending from a proximal end of the tip member to a distal end of the tip member.

Another example medical device delivery system includes an outer shaft having a proximal end region, a distal end region and a lumen extending therein. The delivery system also includes an inner shaft having a proximal end region, a distal end region and a lumen extending therein, wherein the inner shaft extends within at least a portion of the lumen of the outer shaft. The delivery system also includes a tip member disposed the along the distal end region of the outer shaft, the tip member including a stem, a shoulder region, and a projection positioned radially outward of the stem. The delivery system also includes an implantable medical device disposed within the lumen of the outer shaft, wherein the stem is configured to extend into the lumen of the outer shaft, wherein the shoulder region is positioned adjacent a distal end of the outer shaft and wherein the projection extends along an outer surface of the outer shaft.

Alternatively or additionally to any of the embodiments above, wherein at least a portion of the projection covers at least a portion of the distal end region of the outer shaft.

Alternatively or additionally to any of the embodiments above, wherein the projection extends along the outer shaft in a proximal direction.

Alternatively or additionally to any of the embodiments above, wherein a proximal end of the projection is positioned distal to a proximal end of the stem.

Alternatively or additionally to any of the embodiments above, wherein the tip member tapers from the projection to the distal end region of the outer shaft.

Alternatively or additionally to any of the embodiments above, wherein the implantable medical device includes an implantable heart valve.

Alternatively or additionally to any of the embodiments above, wherein the tip member further includes a lumen extending from the proximal end of the tip member to the distal end of the tip member.

An example method for implanting an implantable heart valve at a target site, the method includes advancing an implantable heart valve delivery system to a target site, the delivery system including: an outer shaft having a proximal end region, a distal end region and a lumen extending therein; an inner shaft having a proximal end region, a distal end region and a lumen extending therein, wherein the inner shaft extends within at least a portion of the lumen of the outer shaft; an implantable medical device disposed within the lumen of the outer shaft and a tip member disposed the along the distal end region of the outer shaft, the tip member including a proximal end region and a distal end region, wherein the tip member includes an annular groove positioned adjacent the proximal end region of the tip member, wherein the distal end of the outer shaft is engaged with the annular groove of the tip member. The method also includes retracting the outer shaft relative to the implantable heart valve, wherein retracting the outer shaft uncovers the implantable heart valve.

Alternatively or additionally to any of the embodiments above, wherein the annular groove is proximally facing.

Alternatively or additionally to any of the embodiments above, wherein the annular groove has a first profile and wherein the distal end region of the outer shaft has a second profile, and wherein the first profile is configured to match the second profile.

Alternatively or additionally to any of the embodiments above, wherein the implantable medical device includes an implantable heart valve.

Alternatively or additionally to any of the embodiments above, wherein a portion of the proximal end region of the tip member is configured to extend within at least a portion of the lumen of the outer shaft.

The above summary of some embodiments is not intended to describe each disclosed embodiment or every implementation of the present disclosure. The Figures, and Detailed Description, which follow, more particularly exemplify these embodiments.

### BRIEF DESCRIPTION OF THE DRAWINGS

The disclosure may be more completely understood in consideration of the following detailed description in connection with the accompanying drawings, in which:

FIG. 1 is a side view of an example medical device delivery system;

FIG. 2 is a partial cross-sectional view of a portion of an example medical device delivery system;

FIG. 3 is a partial cross-sectional view of a portion of an example medical device delivery system.

While the disclosure is amenable to various modifications and alternative forms, specifics thereof have been shown by way of example in the drawings and will be described in detail. It should be understood, however, that the intention is not to limit the disclosure to the particular embodiments described. On the contrary, the intention is to cover all modifications, equivalents, and alternatives falling within the spirit and scope of the disclosure.

### DETAILED DESCRIPTION

For the following defined terms, these definitions shall be applied, unless a different definition is given in the claims or elsewhere in this specification.

All numeric values are herein assumed to be modified by the term “about”, whether or not explicitly indicated. The term “about” generally refers to a range of numbers that one of skill in the art would consider equivalent to the recited value (e.g., having the same function or result). In many instances, the terms “about” may include numbers that are rounded to the nearest significant figure.

The recitation of numerical ranges by endpoints includes all numbers within that range (e.g. 1 to 5 includes 1, 1.5, 2, 2.75, 3, 3.80, 4, and 5).

As used in this specification and the appended claims, the singular forms “a”, “an”, and “the” include plural referents unless the content clearly dictates otherwise. As used in this specification and the appended claims, the term “or” is generally employed in its sense including “and/or” unless the content clearly dictates otherwise.

It is noted that references in the specification to “an embodiment”, “some embodiments”, “other embodiments”, etc., indicate that the embodiment described may include one or more particular features, structures, and/or characteristics. However, such recitations do not necessarily mean that all embodiments include the particular features, structures, and/or characteristics. Additionally, when particular features, structures, and/or characteristics are described in connection with one embodiment, it should be understood that such features, structures, and/or characteristics may also be used connection with other embodiments whether or not explicitly described unless clearly stated to the contrary.

The following detailed description should be read with reference to the drawings in which similar elements in different drawings are numbered the same. The drawings, which are not necessarily to scale, depict illustrative embodiments and are not intended to limit the scope of the invention.

Diseases and/or medical conditions that impact the cardiovascular system are prevalent throughout the world. Traditionally, treatment of the cardiovascular system was often conducted by directly accessing the impacted part of the body. For example, treatment of a blockage in one or more of the coronary arteries was traditionally treated using coronary artery bypass surgery. As can be readily appreciated, such therapies are rather invasive to the patient and require significant recovery times and/or treatments. More recently, less invasive therapies have been developed. For example, therapies have been developed which allow a blocked coronary artery to be accessed and treated via a percutaneous catheter (e.g., angioplasty). Such therapies have gained wide acceptance among patients and clinicians.

Some relatively common medical conditions may include or be the result of inefficiency, ineffectiveness, or complete failure of one or more of the valves within the heart. For example, failure of the aortic valve or the mitral valve can have a serious effect on a human and could lead to serious health condition and/or death if not dealt with properly. Treatment of defective heart valves poses other challenges in that the treatment often requires the repair or outright replacement of the defective valve. Such therapies may be highly invasive to the patient. Disclosed herein are medical devices that may be used for delivering a medical device to a portion of the cardiovascular system in order to diagnose, treat, and/or repair the system. At least some of the medical devices disclosed herein may be used to deliver and implant a replacement heart valve (e.g., a replacement aortic valve, replacement mitral valve, etc.). In addition, the devices disclosed herein may deliver the replacement heart valve percutaneously and, thus, may be much less invasive to the patient. The devices disclosed herein may also provide a number of additional desirable features and benefits as described in more detail below.

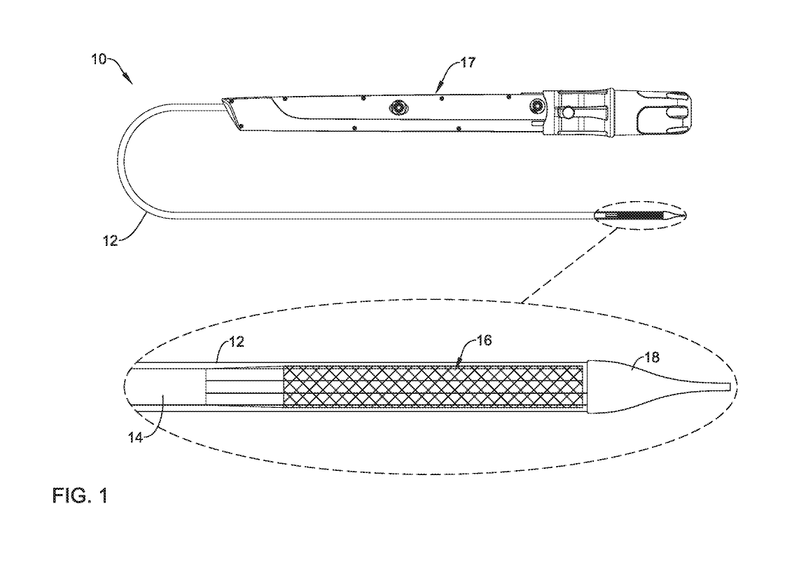

The figures illustrate selected components and/or arrangements of a medical device system 10, shown schematically in FIG. 1 for example. It should be noted that in any given figure, some features of the medical device system 10 may not be shown, or may be shown schematically, for simplicity. Additional details regarding some of the components of the medical device system 10 may be illustrated in other figures in greater detail. A medical device system 10 may be used to deliver and/or deploy a variety of medical devices to a number of locations within the anatomy. In at least some embodiments, the medical device system 10 may include a replacement heart valve delivery system (e.g., a replacement aortic valve delivery system) that can be used for percutaneous delivery of a medical implant 16 (shown in the detailed view of FIG. 1), such as a replacement/prosthetic heart valve. This, however, is not intended to be limiting as the medical device system 10 may also be used for other interventions including valve repair, valvuloplasty, delivery of an implantable medical device (e.g., such as a stent, graft, etc.), and the like, or other similar interventions.

The medical device system 10 may generally be described as a catheter system that includes an outer shaft 12, an inner shaft 14 extending at least partially through a lumen of the outer shaft 12, and a medical implant 16 (e.g., a replacement heart valve implant) which may be coupled to the inner shaft 14 and disposed within a lumen of the outer shaft 12 during delivery of the medical implant 16. Additionally, FIG. 1 illustrates that a tip member 18 may be disposed at the distal end region of medical device system 10.

In some embodiments, a medical device handle 17 may be disposed at a proximal end of the outer shaft 12 and/or the inner shaft 14 and may include one or more actuation mechanisms associated therewith. In other words, one or more tubular members (e.g., the outer shaft 12, the inner shaft 14, etc.) may extend distally from the medical device handle 17. In general, the medical device handle 17 may be designed to manipulate the position of the outer shaft 12 relative to the inner shaft 14 and/or aid in the deployment of the medical implant 16.

In use, the medical device system 10 may be advanced percutaneously through the vasculature to a position adjacent to an area of interest and/or a treatment location. For example, in some embodiments, the medical device system 10 may be advanced through the vasculature to a position adjacent to a defective native valve (e.g., aortic valve, mitral valve, etc.). Alternative approaches to treat a defective aortic valve and/or other heart valve(s) are also contemplated with the medical device system 10.

During delivery, the medical implant 16 may be generally disposed in an elongated and low profile “delivery” configuration within the lumen and/or a distal end of the outer shaft 12. For example, as shown in FIG. 1, the medical implant may be positioned between the outer shaft 12 and the inner shaft 14. In other words, the medical implant 16 may be covered by the outer shaft 12 while positioned in an “undeployed” configuration. Further, once positioned at a target site, the outer shaft 12 may be retracted relative to the medical implant 16 and/or the inner shaft 14 to expose the medical implant 16. In some instances, the medical implant 16 may be self-expanding such that exposure of the medical implant 16 may deploy the medical implant 16. Alternatively, the medical implant 16 may be expanded/deployed using the medical device handle 17 in order to translate the medical implant 16 into a generally shortened and larger profile “deployed” configuration suitable for implantation within the anatomy. When the medical implant 16 is suitably deployed within the anatomy, the medical device system 10 may be disconnected, detached, and/or released from the medical implant 16 and the medical device system 10 can be removed from the vasculature, leaving the medical implant 16 in place in a “released” configuration.

It can be appreciated that during delivery and/or deployment of an implantable medical device (e.g., the medical implant 16), portions of the medical device system (e.g., the medical device system 10) may be required to be advanced through tortuous and/or narrow body lumens. Therefore, it may be desirable to utilize components and design medical delivery systems (e.g., such as the medical device system 10 and/or other medical devices) that improve the ability of the medical device to track efficiently through the vasculature while maintaining a low profile and flexibility of the system as a whole. For example, as medical device system is advanced through the body, some body vessel may include tight curves through which the device may be required to traverse. It can be appreciated that as the outer shaft 12 advances through a tight curve, its profile may flex from a generally circular cross-section to a more oval-shaped cross-section. In other words, the cross-sectional shaped of the outer shaft 12 may “ovalize” as it is advanced through the body. This “ovalization” of the outer shaft 12 may be limit the medical device system 10 from efficiently advancing through the body lumen. Therefore, in some instances it may be desirable to design the tip member 18 such that it prevents the outer shaft 12 from flexing from a circular shape to an ovular shape.

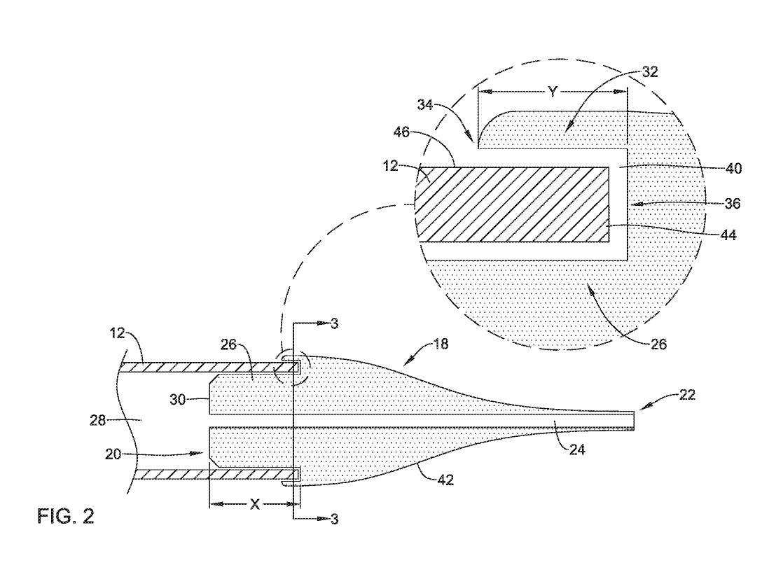

FIG. 2 is a partial cross-sectional view of the tip member 18 and outer shaft 12 of the medical device system 10 (for simplicity, the inner shaft 14 and the medical implant 16 are not shown in FIG. 2). FIG. 2 shows that tip member 18 may include a proximal end region 20 and a distal end region 22. Tip member 18 may further include a lumen 24 extending within tip member 18 from the proximal end region 20 to the distal end region 22. In some instances, lumen 24 may be utilized to pass a guidewire therethrough.

FIG. 2 further illustrates that tip member 18 may include a stem portion 26. Stem portion 26 may include a proximal end 30. As shown in FIG. 2, stem portion 26 may extend into the lumen 28 of the outer shaft 12. For example, FIG. 2 illustrates that stem portion 26 may extend a distance “X” into the lumen 28 of the outer shaft 12. The distance “X” may be the distance from the proximal end 30 of the stem portion 26 to a distal end 44 of the outer shaft 12.

FIG. 2 further illustrates that in some instances, the tip member 18 may include a proximally-extending projection 32. Projection 32 may be positioned radially outward of the outer shaft 12. The detailed view of FIG. 2 illustrates that the projection 32 may be disposed along an outer surface 46 of the outer shaft 12 (e.g., the projection may “cover” a portion of the outer shaft 12). For example, FIG. 2 illustrates that the projection 32 may extend along the outer surface 46 of the outer shaft 12 a distance depicted as “Y.” Further, a proximal end 34 of the projection 32 may be positioned distally of the proximal end 30 of the stem 26.

Additionally, the detailed view of FIG. 2 shows that tip member 18 may include a shoulder region 36 extending from the projection 32 to a distal portion of the stem portion 26. In some instances, the shoulder region 36 may extend substantially perpendicular to the projection 32 and the stem 26.

It can further be appreciated from FIG. 2 that the combination of the projection 32, the shoulder region 36 and the distal portion of the stem portion 26 may define an “annular groove” 40. The annular groove may be defined as a “channel” that extends circumferentially around the tip member 18. It can be appreciated that the annular groove may have a depth which is substantially equivalent to the length of projection 32. In other words, the annular groove 40 may have a depth depicted as “Y” in FIG. 2. Additionally, it can be appreciated that the annular groove 40 may include a profile that substantially matches (e.g., “mates” with) the profile of the distal end of the outer shaft 12. While FIG. 2 illustrates that the profile of the annular groove 40 and the distal end of the outer shaft is substantially rectangular, other profiles are contemplated. Further, it can be appreciated that the annular groove 40 may be “proximally facing.” As described herein, “proximally facing” may mean that the annular groove 40 is open and/or accessible from the proximal direction. In other words, the opening which defines the annular groove 40 opens away from the distal end of the distal tip member 18.

In some instances, it may be desirable to insert a portion of the outer shaft 12 into the annular groove 40. FIG. 2 illustrates that a distal end region of outer shaft 12 is inserted into the annular groove 40. For example, the distal end region of the outer shaft 12 may be inserted a distance “Y” into the annular groove. As shown in FIG. 2, when the outer shaft 12 is inserted into the annular groove a distance “Y,” the distal end 44 of the outer shaft 12 may be adjacent the shoulder region 36 of the tip member 18. It can further be appreciated that the entire circumference of the outer shaft 12 may inserted into the annular groove (which, as discussed above, extends circumferentially around the tip member 18).

As discussed above, it may be desirable to design the tip member 18 such that it prevents the outer shaft 12 from flexing from a circular shape to an ovular shape. It can be appreciated that disposing a portion of the outer shaft 12 into the annular groove 40 of the tip member 18 may limit the outer shaft 12 from shifting from a generally circularly shape to an ovular shape. In other words, inserting the distal region of the outer shaft 12 into the annular groove may allow the tip member 18 (in particular, the projection 32) to hold the outer surface 46 of the distal end region of the outer shaft 12 in a circular shape. The projection 32 prevents the outer surface 46 of the distal end of the outer shaft 12 from flexing away from a generally circular shape. It can be appreciated that the outer shaft 12 may extend into the annular groove 40 while not being rigidly fixed to the tip member 18. In other words, the tip member may maintain the distal end region of the outer shaft 12 in a circular configuration while medical device system 10 is tracked through the anatomy, even though the distal end region of the outer shaft 12 is no rigidly fixed to the tip member 18.

FIG. 2 further illustrates that the tip member 18 may include a taper portion 42. The tapered portion may extend from the projection 32 to the distal end region 22 of the tip member 18. As illustrated in FIG. 2, the outer diameter of the tip member 18 may decrease in a proximal-to-distal direction along the tapered portion 42 of the tip member 18.

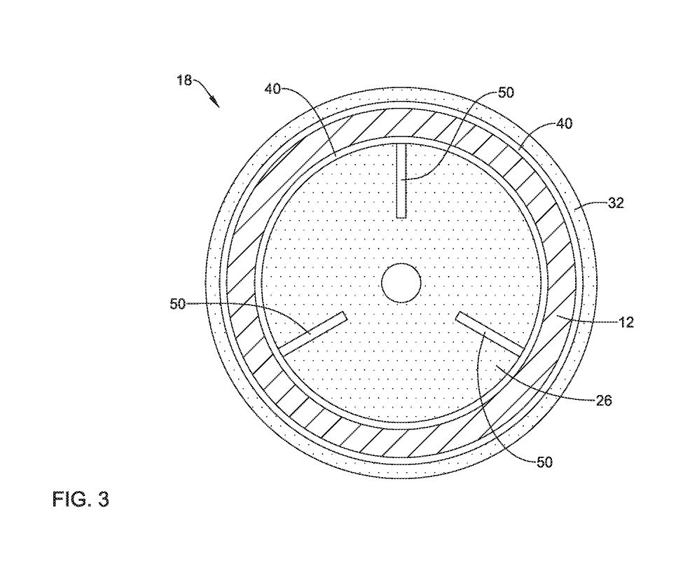

FIG. 3 is a cross-sectional view along line 3-3 of FIG. 2. FIG. 3 shows the outer shaft 12 positioned between the stem portion 26 and the projection 32. In other words, FIG. 3 shows that the outer shaft (when inserted into the annular groove 40, as discussed with respect to FIG. 2) positioned radially outward of the stem member 26 and radially inward of the projection 32. Further, FIG. 3 shows that the annular groove 40 extends circumferentially around the tip member 18 and that the outer shaft 12 may be inserted into the annular groove 40 circumferentially around the tip member 18. Additionally, it is contemplated that in some instances the stem 26 may be free of grooves 50.

The materials that can be used for the various components of the medical devices and/or system 10 disclosed herein may include those commonly associated with medical devices. However, this is not intended to limit the devices and methods described herein, as the discussion may be applied to other components of the medical devices and/or systems 10 disclosed herein including the various shafts, liners, components described relative thereto.

The medical device system 10 and components thereof may be made from a metal, metal alloy, polymer (some examples of which are disclosed below), a metal-polymer composite, ceramics, combinations thereof, and the like, or other suitable material. Some examples of suitable polymers may include polytetrafluoroethylene (PTFE), ethylene tetrafluoroethylene (ETFE), fluorinated ethylene propylene (FEP), polyoxymethylene (POM, for example, DELRIN® available from DuPont), polyether block ester, polyurethane (for example, Polyurethane 85A), polypropylene (PP), polyvinylchloride (PVC), polyether-ester (for example, ARNITEL® available from DSM Engineering Plastics), ether or ester based copolymers (for example, butylene/poly(alkylene ether) phthalate and/or other polyester elastomers such as HYTREL® available from DuPont), polyamide (for example, DURETHAN® available from Bayer or CRISTAMID® available from Elf Atochem), elastomeric polyamides, block polyamide/ethers, polyether block amide (PEBA, for example available under the trade name PEBAX®), ethylene vinyl acetate copolymers (EVA), silicones, polyethylene (PE), high density polyethylene (HDPE), polyester, Marlex high-density polyethylene, Marlex low-density polyethylene, linear low density polyethylene (for example REXELL®), ultra-high molecular weight (UHMW) polyethylene, polypropylene, polybutylene terephthalate (PBT), polyethylene terephthalate (PET), polytrimethylene terephthalate, polyethylene naphthalate (PEN), polyetheretherketone (PEEK), polyimide (PI), polyetherimide (PEI), polyphenylene sulfide (PPS), polyphenylene oxide (PPO), poly paraphenylene terephthalamide (for example, KEVLAR®), polysulfone, nylon, nylon-12 (such as GRILAMID® available from EMS American Grilon), perfluoro(propyl vinyl ether) (PFA), ethylene vinyl alcohol, polyolefin, polystyrene, epoxy, polyvinylidene chloride (PVdC), poly(styrene-b-isobutylene-b-styrene) (for example, SIBS and/or SIBS 50A), polycarbonates, ionomers, biocompatible polymers, other suitable materials, or mixtures, combinations, copolymers thereof, polymer/metal composites, and the like. In some embodiments the sheath can be blended with a liquid crystal polymer (LCP).

Some examples of suitable metals and metal alloys include stainless steel, such as 304V, 304L, and 316LV stainless steel; mild steel; nickel-titanium alloy such as linear-elastic and/or super-elastic nitinol; other nickel alloys such as nickel-chromium-molybdenum alloys (e.g., UNS: N06625 such as INCONEL® 625, UNS: N06022 such as HASTELLOY® C-22®, UNS: N10276 such as HASTELLOY® C276®, other HASTELLOY® alloys, and the like), nickel-copper alloys (e.g., UNS: N04400 such as MONEL® 400, NICKELVAC® 400, NICORROS® 400, and the like), nickel-cobalt-chromium-molybdenum alloys (e.g., UNS: R30035 such as MP35-N® and the like), nickel-molybdenum alloys (e.g., UNS: N10665 such as HASTELLOY® ALLOY B2®), other nickel-chromium alloys, other nickel-molybdenum alloys, other nickel-cobalt alloys, other nickel-iron alloys, other nickel-copper alloys, other nickel-tungsten or tungsten alloys, and the like; cobalt-chromium alloys; cobalt-chromium-molybdenum alloys (e.g., UNS: R30003 such as ELGILOY®, PHYNOX®, and the like); platinum enriched stainless steel; titanium; combinations thereof; and the like; or any other suitable material.

In at least some embodiments, portions or all of the medical device system 10 and components thereof may also be doped with, made of, or otherwise include a radiopaque material. Radiopaque materials are understood to be materials capable of producing a relatively bright image on a fluoroscopy screen or another imaging technique during a medical procedure. This relatively bright image aids the user of the medical device 10 in determining its location. Some examples of radiopaque materials can include, but are not limited to, gold, platinum, palladium, tantalum, tungsten alloy, polymer material loaded with a radiopaque filler, and the like. Additionally, other radiopaque marker bands and/or coils may also be incorporated into the design of the medical device 10 to achieve the same result.

In some embodiments, a degree of Magnetic Resonance Imaging (Mill) compatibility is imparted into the medical device system 10 and components thereof. For example, the medical device 10 may include a material that does not substantially distort the image and create substantial artifacts (e.g., gaps in the image). Certain ferromagnetic materials, for example, may not be suitable because they may create artifacts in an MRI image. The medical device 10 may also be made from a material that the MM machine can image. Some materials that exhibit these characteristics include, for example, tungsten, cobalt-chromium-molybdenum alloys (e.g., UNS: R30003 such as ELGILOY®, PHYNOX®, and the like), nickel-cobalt-chromium-molybdenum alloys (e.g., UNS: R30035 such as MP35-N® and the like), nitinol, and the like, and others.

It should be understood that this disclosure is, in many respects, only illustrative. Changes may be made in details, particularly in matters of shape, size, and arrangement of steps without exceeding the scope of the disclosure. This may include, to the extent that it is appropriate, the use of any of the features of one example embodiment being used in other embodiments. The disclosure's scope is, of course, defined in the language in which the appended claims are expressed.

What is claimed is:

1. A medical device delivery system, comprising:
an outer shaft having a proximal end region, a distal end region and a lumen extending therein;
an inner shaft having a proximal end region, a distal end region and a lumen extending therein, wherein the inner shaft extends within at least a portion of the lumen of the outer shaft; and
a tip member disposed the along the distal end region of the outer shaft, the tip member including a proximal end region and a distal end region;
wherein the lumen of the outer shaft is configured to position an implantable medical device therein;
wherein the tip member includes an annular groove positioned adjacent the proximal end region of the tip member.

2. The delivery system of claim 1, wherein at least a portion of the distal end region of the outer shaft is designed to engage the annular groove.

3. The delivery system of claim 1, wherein the annular groove is proximally facing.

4. The delivery system of claim 1, wherein the annular groove has a first profile and wherein the distal end region of the outer shaft has a second profile, and wherein the first profile is configured to match the second profile.

5. The delivery system of claim 1, wherein a portion of the tip covers at least a portion of the distal end region of the outer shaft.

6. The delivery system of claim 1, wherein a portion of the proximal end region of the tip member is configured to extend within at least a portion of the lumen of the outer shaft.

7. The delivery system of claim 1, wherein the implantable medical device includes an implantable heart valve.

8. The delivery system of claim 1, wherein the tip member further includes a lumen extending from a proximal end of the tip member to a distal end of the tip member.

9. A medical device delivery system, comprising:
an outer shaft having a proximal end region, a distal end region and a lumen extending therein;
an inner shaft having a proximal end region, a distal end region and a lumen extending therein, wherein the inner shaft extends within at least a portion of the lumen of the outer shaft; and
a tip member disposed the along the distal end region of the outer shaft, the tip member including a stem, a shoulder region, and a projection positioned radially outward of the stem;
an implantable medical device disposed within the lumen of the outer shaft;
wherein the stem is configured to extend into the lumen of the outer shaft;
wherein the shoulder region is positioned adjacent a distal end of the outer shaft;
wherein the projection extends along an outer surface of the outer shaft.

10. The delivery system of claim 9, wherein at least a portion of the projection covers at least a portion of the distal end region of the outer shaft.

11. The delivery system of claim 9, wherein the projection extends along the outer shaft in a proximal direction.

12. The delivery system of claim 9, wherein a proximal end of the projection is positioned distal to a proximal end of the stem.

13. The delivery system of claim 9, wherein the tip member tapers from the projection to the distal end region of the outer shaft.

14. The delivery system of claim 9, wherein the implantable medical device includes an implantable heart valve.

15. The delivery system of claim 9, wherein the tip member further includes a lumen extending from the proximal end of the tip member to the distal end of the tip member.

16. A method for implanting an implantable heart valve at a target site, the method comprising:
advancing an implantable heart valve delivery system to a target site, the delivery system including:
an outer shaft having a proximal end region, a distal end region and a lumen extending therein;
an inner shaft having a proximal end region, a distal end region and a lumen extending therein, wherein the inner shaft extends within at least a portion of the lumen of the outer shaft;
an implantable medical device disposed with the lumen of the outer shaft; and
a tip member disposed the along the distal end region of the outer shaft, the tip member including a proximal end region and a distal end region, wherein the tip member includes an annular groove positioned adjacent the proximal end region of the tip member;
wherein the distal end of the outer shaft is engaged with the annular groove of the tip member;
retracting the outer shaft relative to the implantable heart valve, wherein retracting the outer shaft uncovers the implantable heart valve.

17. The delivery system of claim 16, wherein the annular groove is proximally facing.

18. The delivery system of claim 16, wherein the annular groove has a first profile and wherein the distal end region of the outer shaft has a second profile, and wherein the first profile is configured to match the second profile.

19. The delivery system of claim 16, wherein the implantable medical device includes an implantable heart valve.

20. The delivery system of claim 16, wherein a portion of the proximal end region of the tip member is configured to extend within at least a portion of the lumen of the outer shaft.
